(19) United States
(12) Patent Application Publication
(10) Pub. No.: US 2023/0087560 A1
(43) Pub. Date: Mar. 23, 2023

(54) HYDROFLUORETHERS AND METHODS OF USING SAME

(71) Applicant: 3M INNOVATIVE PROPERTIES COMPANY, St. Paul, MN (US)

(72) Inventors: William M. Lamanna, Stillwater, MN (US); Klaus Hintzer, Kastl (DE); Markus Hirschberg, Muhldorf (DE); David J. Lundberg, Cottage Grove, MN (US)

(21) Appl. No.: 17/791,948

(22) PCT Filed: Jan. 11, 2021
(86) PCT No.: PCT/IB2021/050166

§ 371 Date: Jul. 11, 2022

Related U.S. Application Data
(60) Provisional application No. 62/961,365, filed on Jan. 15, 2020.

(51) Int. Cl. C07C 43/12; H01L 21/67; F28C 3/04; H05K 7/20

(52) U.S. Cl. CPC C07C 43/12; H01L 21/67; F28C 3/04; H05K 7/20

(57) ABSTRACT

A compound having structural formula (II): where R² is H, CH₃, CF₃, CH₂CF₂CF₂H, or CH₂CF₂CF₂CF₂H; where R²f is a perfluoroalkyl group having 1-4 carbon atoms, optionally comprising either or both of a catenated nitrogen heteroatom and a catenated oxygen heteroatom; where R²f′ is CF₃ or CF₂CF₃; and where R²f″ is CF₃ or CF₂CF₃; with the proviso that when R²f′ is CF₃, then R²f″ is CF₃, and when R² is H or CH₃, then R²f is not CF₃.

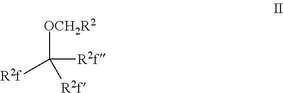

Various hydrofluoroethers and their uses are described in, for example, U.S. Pat. Nos. 5,718,293, 5,925,611, and 6,046,368.

### FIELD

The present disclosure relates to hydrofluoroethers, working fluids that include the same, systems and devices that include the same, and methods of using the same.

### DETAILED DESCRIPTION

In view of an increasing demand for environmentally friendly chemical compounds, it is recognized that there exists an ongoing need for new working fluids that provide reductions in environmental impact while still meeting or exceeding the performance requirements (e.g., nonflammability, solvency, stability, and operating temperature range) of a variety of different applications (e.g., heat transfer, immersion cooling, foam blowing agents, solvent cleaning, and deposition coating solvents), and that can be manufactured cost-effectively.

Generally, the present disclosure relates to hydrofluoroether compounds that contain a tertiary perfluoroalkyl group bound to oxygen. These hydrofluoroether compounds exhibit surprisingly higher hydrolytic and base stability compared to related hydrofluoroether compounds that do not contain a tertiary perfluoroalkyl group bound to oxygen. Furthermore, the hydrofluoroether compounds of the present disclosure exhibit shorter atmospheric lifetimes and lower global warming potentials than comparable fluorinated compounds useful as working fluids (e.g., perfluorinated hydrocarbons, or hydrofluorocarbons, or hydrofluoroether compounds that do not contain a tertiary perfluoroalkylgroup bound to oxygen).

As used herein, “catenated heteroatom” means an atom other than carbon (for example, oxygen, nitrogen, or sulfur) that is bonded to at least two carbon atoms in a carbon chain (linear or branched or within a ring) so as to form a carbon-heteroatom-carbon linkage.

As used herein, “fluoro-” (for example, in reference to a group or moiety, such as in the case of “fluoroalkene” or “fluoroalkenyl” or “fluoroalkane” or “fluoroalkyl” or “fluorocarbon”) or “fluorinated” means (i) partially fluorinated such that there is at least one carbon-bonded hydrogen atom, or (ii) perfluorinated.

As used herein, “perfluoro-” (for example, in reference to a group or moiety, such as in the case of “fluoroalkene” or “fluoroalkenyl” or “fluoroalkane” or “fluoroalkyl” or “fluorocarbon”) or “perfluorinated” means completely fluorinated such that, except as may be otherwise indicated, there are no carbon-bonded hydrogen atoms replaceable with fluorine.

As used herein, “alkyl” means a molecular fragment comprised of a valence-saturated carbon-based skeleton (i.e., derived from an alkane), which may be linear, branched or cyclic.

As used herein, “alkenyl” means a molecular fragment comprised of a carbon-base skeleton, which contains at least one carbon-carbon double bond (i.e., derived from alkene, diene, etc.); alkenyl fragments may be linear, branched or cyclic.

As used herein, the singular forms “a”, “an”, and “the” include plural referents unless the content clearly dictates otherwise. As used in this specification and the appended embodiments, the term “or” is generally employed in its sense including “and/or” unless the content clearly dictates otherwise.

As used herein, the recitation of numerical ranges by endpoints includes all numbers subsumed within that range (e.g., 1 to 5 includes 1, 1.5, 2, 2.75, 3, 3.8, 4, and 5).

Unless otherwise indicated, all numbers expressing quantities or ingredients, measurement of properties and so forth used in the specification and embodiments are to be understood as being modified in all instances by the term “about.” Accordingly, unless indicated to the contrary, the numerical parameters set forth in the foregoing specification and attached listing of embodiments can vary depending upon the desired properties sought to be obtained by those skilled in the art utilizing the teachings of the present disclosure. At the very least, and not as an attempt to limit the application of the doctrine of equivalents to the scope of the claimed embodiments, each numerical parameter should be construed in light of the number of reported significant digits and by applying ordinary rounding techniques.

In some embodiments, the present disclosure is directed to a compound having structural formula (I)

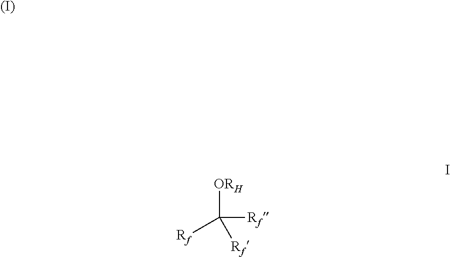

where R_{H} is CH₃, CH₂CH₃, or a partially fluorinated alkyl group having 1-5 or 1-2 carbon atoms;

where Rf is a perfluoroalkyl group having 1-9, 4-8, or 5-8 carbon atoms, optionally comprising either or both of a catenated nitrogen heteroatom and a catenated oxygen heteroatom, and optionally comprising a 5- or 6-membered ring; and

where Rf′ and Rf″ are, independently, perfluoroalkyl groups having 1-2 carbon atoms.

In some embodiments, R_{H} may be CH₃ or CH₂CH₃. In some embodiments, R_{H} may be a partially fluorinated alkyl group having 1-5 carbon atoms.

In some embodiments at least one of R_{f}′ and R_{f}″ are perfluoroalkyl groups having 2 carbon atoms (i.e., perfluoroethylgroups).

In some embodiments, both R_{f}′ and R_{f}″ are perfluoroalkyl groups having 2 carbon atoms (i.e., perfluoroethylgroups).

In any of the above described embodiments, Rf may comprise either or both of a catenated nitrogen heteroatom and a catenated oxygen heteroatom. In any of the above described embodiments, Rf may comprise a catenated nitrogen heteroatom. In any of the above described embodiments, Rf may comprise a catenated oxygen heteroatom. In any of the above described embodiments, Rf may comprise a catenated nitrogen heteroatom and a catenated oxygen heteroatom.

As will be discussed in greater detail below, a subset of compounds within structural formula (I) may be manufactured particularly cost effectively. Such compounds may have a structural formula (II):

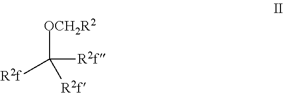

where R² is H, CH₃, CF₃, CH₂CF₂CF₂H, or CH₂CF₂CF₂CF₂CF₂H
where R²f is a perfluoroalkyl group having 1-4 or 2-4 carbon atoms, optionally comprising either or both of a catenated nitrogen heteroatom and a catenated oxygen heteroatom;
where R²f is CF₃ or CF₂CF₃; and
where R²f′ is CF₃ or CF₂CF₃;
with the proviso that when R²f′ is CF₃, R²f″ is CF₃, and when R² is H or CH₃, then R²f is not CF³.

In some embodiments, R² is H or CH₃.

In various embodiments, representative examples of the compounds of structural formula (I) and (II) include the following:

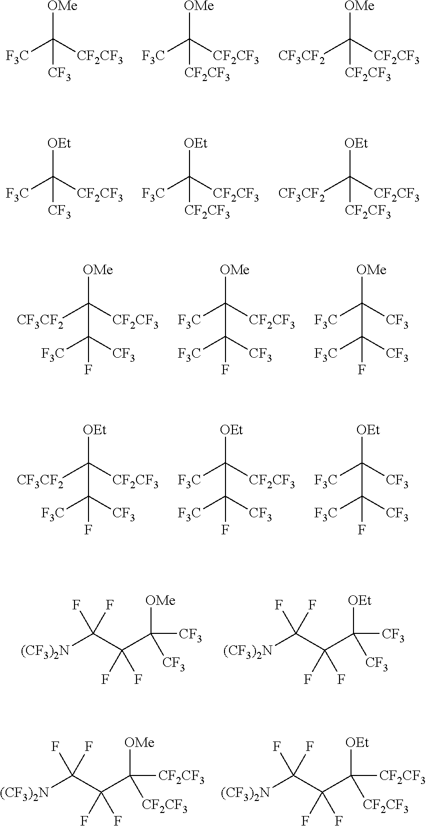

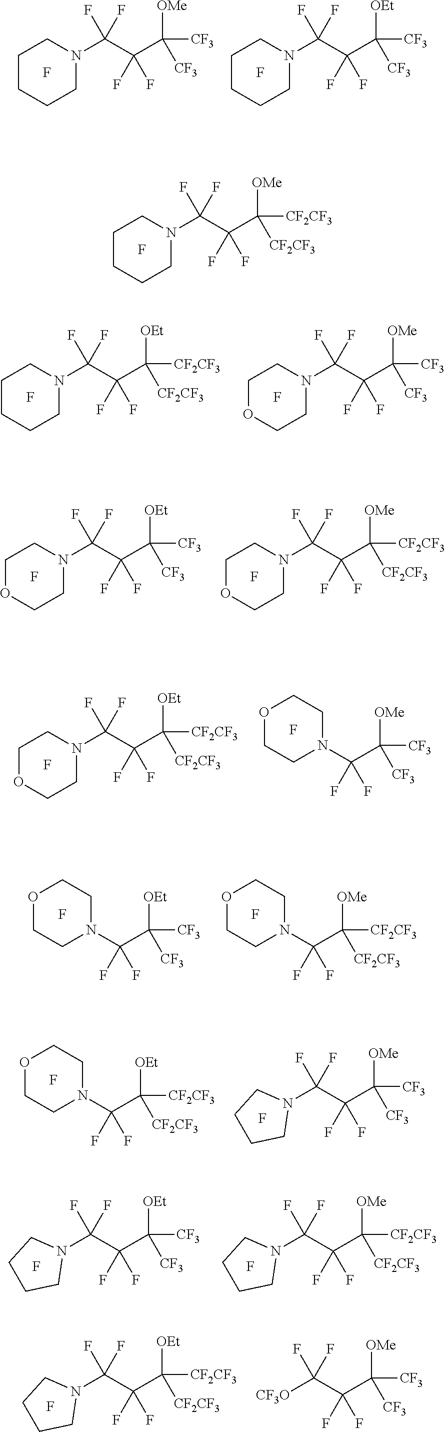

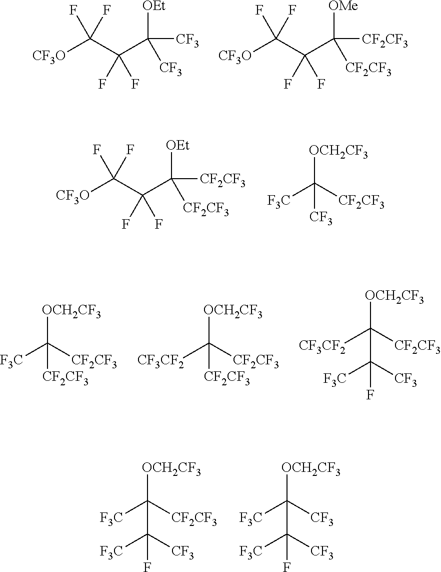

where “Me” is a methyl group (CH₃) and “Et” is an ethyl group (CH₂CH₃).

In some embodiments, the fluorine content in the compounds of the present disclosure (i.e., the compounds having Structural Formula (I) or (II)) may be sufficient to make the compounds non-flammable according to ASTM D-3278-96 e-1 test method (“Flash Point of Liquids by Small Scale Closed Cup Apparatus”).

In some embodiments, the compounds of the present disclosure (i.e., the tert-fluoroalkyl-containing hydrofluoroethers of structural formulas (I) and (II)) may be useful over a broad operating temperature range. In this regard, in some embodiments, the compounds of the present disclosure may have a boiling point of no lower than 30, 40, 50, 60, 70, 80, or 90 degrees Celsius and no higher than 290, 270, 250, 230, 210, 190, 170, 150 130, 120, 110, 100, 90, or 80 degrees Celsius.

In some embodiments, the compounds of the present disclosure may be hydrophobic, relatively chemically unreactive, and thermally stable. The fluorinated compounds may have a low environmental impact. In this regard, the fluorinated compounds of the present disclosure may have a global warming potential (GWP, 100 yr ITH) of less than 500, 300, 200, 100, 50, 10, or less than 1. As used herein, GWP is a relative measure of the global warming potential of a compound based on the structure of the compound. The GWP of a compound, as defined by the Intergovernmental Panel on Climate Change (IPCC) in 1990 and updated in 2007, is calculated as the warming due to the release of 1 kilogram of a compound relative to the warming due to the release of 1 kilogram of CO₂ over a specified integration time horizon (ITH).

$${GWP}_{i} ( {t}^{′} ) = \frac{{\int }_{0}^{ITH} {a}_{i} [ C ( t ) ] dt}{{\int }_{0}^{ITH} {a}_{{CO}_{2}} [ {C}_{{CO}_{2}} ( t ) ] dt} = \frac{{\int }_{0}^{ITH} {a}_{i} {C}_{oi} {e}^{- t / {τ}_{i}} dt}{{\int }_{0}^{ITH} {a}_{{CO}_{2}} [ {C}_{{CO}_{2}} ( t ) ] dt}$$

In this equation ai is the radiative forcing per unit mass increase of a compound in the atmosphere (the change in the flux of radiation through the atmosphere due to the IR absorbance of that compound), C is the atmospheric concentration of a compound, τ is the atmospheric lifetime of a compound, t is time, and i is the compound of interest. The commonly accepted ITH is 100 years representing a compromise between short-term effects (20 years) and longer-term effects (500 years or longer). The concentration of an organic compound, i, in the atmosphere is assumed to follow pseudo first order kinetics (i.e., exponential decay). The concentration of CO₂ over that same time interval incorporates a more complex model for the exchange and removal of CO₂ from the atmosphere (the Bern carbon cycle model).

In some embodiments, the compounds of the present disclosure can be prepared from their respective perfluorinated acid fluorides or ketones in combination with tetrafluoroethylene (TFE) or perfluoroalkyl trimethyl silane in the presence of a metal fluoride catalyst/reagent ([M]F) such as KF or CsF in an aprotic organic solvent (e.g., diglyme, tetraglyme, N,N-dimethylformamide, or N-methylpyrrolidine). The metal perfluoroalkoxide intermediate may then be quenched by the addition of electrophile R_{H}-X (e.g., iodomethane, bromomethane, dimethylsulfate, iodoethane, bromoethane, diethylsulfate, 2,2,2-trifluoroethyl trifluoromethanesulfonate, 2,2,3,3,3-pentafluoropropyl trifluoromethanesulfonate, 2,2,3,3,4,4,4-heptafluorobutyl trifluoromethanesulfonate, and 2,2,2-trifluoroethyl 1,1,2,2,3,3,4,4,4-nonafluorobutane-1-sulfonate) to afford the desired composition. Readily available and low cost fluorochemical building blocks such as perfluorinated acid fluorides, perfluoroketones, and tetrafluoroethylene (TFE) render the compositions of this disclosure cost effective working fluids. Furthermore, the use of inexpensive fluoride salts such as KF and alkylating reagents (e.g., dimethyl- and diethylsulfate) further support the low-cost synthesis of the compounds of the present disclosure.

In some embodiments, the present disclosure is further directed to working fluids that include one or more of the above-described compounds as a major component. For example, the working fluids may include at least 25%, at least 50%, at least 70%, at least 80%, at least 90%, at least 95%, or at least 99% by weight of the above-described compounds, based on the total weight of the working fluid. In addition to the compounds of the present disclosure, the working fluids may include a total of up to 75%, up to 50%, up to 30%, up to 20%, up to 10%, or up to 5% by weight of one or more of the following components: alcohols, ethers, alkanes, alkenes, haloalkanes, perfluorocarbons, perfluorinated tertiary amines, perfluoroethers, cycloalkanes, esters, ketones, oxiranes, aromatics, siloxanes, hydrochlorocarbons, hydrochlorofluorocarbons, hydrofluorocarbons, hydrochloroolefins, hydrochlorofluoroolefins, sulfones, or mixtures thereof, based on the total weight of the working fluid. Such additional components can be chosen to modify or enhance the properties of a composition for a particular use.

In some embodiments, the compounds of the present disclosure (or working fluids containing the same) can be used in various applications as heat transfer agents (for example, for the cooling or heating of integrated circuit tools in the semiconductor industry, including tools such as dry etchers, integrated circuit testers, photolithography exposure tools (steppers), ashers, chemical vapor deposition equipment, automated test equipment (probers), physical vapor deposition equipment (e.g. sputterers), and vapor phase soldering fluids, and thermal shock fluids).

In some embodiments, the present disclosure is further directed to an apparatus for heat transfer that includes a device and a mechanism for transferring heat to or from the device. The mechanism for transferring heat may include a heat transfer or working fluid that includes one or more compounds of the present disclosure.

The provided apparatus for heat transfer may include a device. The device may be a component, work-piece, assembly, etc. to be cooled, heated or maintained at a predetermined temperature or temperature range. Such devices include electrical components, mechanical components and optical components. Examples of devices of the present disclosure include, but are not limited to microprocessors, wafers used to manufacture semiconductor devices, power control semiconductors, electrical distribution switch gear, power transformers, circuit boards, multi-chip modules, packaged and unpackaged semiconductor devices, lasers, chemical reactors, fuel cells, heat exchangers, and electrochemical cells. In some embodiments, the device can include a chiller, a heater, or a combination thereof.

In yet other embodiments, the devices can include electronic devices, such as processors, including microprocessors. As these electronic devices become more powerful, the amount of heat generated per unit time increases. Therefore, the mechanism of heat transfer plays an important role in processor performance. The heat-transfer fluid typically has good heat transfer performance, good electrical compatibility (even if used in “indirect contact” applications such as those employing cold plates), as well as low toxicity, low (or non-) flammability and low environmental impact. Good electrical compatibility requires that the heat-transfer fluid candidate exhibit high dielectric strength, high volume resistivity, and poor solvency for polar materials. Additionally, the heat-transfer fluid should exhibit good mechanical compatibility, that is, it should not affect typical materials of construction in an adverse manner, and it should have a low pour point and low viscosity to maintain fluidity during low temperature operation.

The provided apparatus may include a mechanism for transferring heat. The mechanism may include a heat transfer fluid. The heat transfer fluid may include one or more fluorinated aromatics of the present disclosure. Heat may be transferred by placing the heat transfer mechanism in thermal contact with the device. The heat transfer mechanism, when placed in thermal contact with the device, removes heat from the device or provides heat to the device, or maintains the device at a selected temperature or temperature range. The direction of heat flow (from device or to device) is determined by the relative temperature difference between the device and the heat transfer mechanism.

The heat transfer mechanism may include facilities for managing the heat-transfer fluid, including, but not limited to pumps, valves, fluid containment systems, pressure control systems, condensers, heat exchangers, heat sources, heat sinks, refrigeration systems, active temperature control systems, and passive temperature control systems.

Examples of suitable heat transfer mechanisms include, but are not limited to, temperature-controlled wafer chucks in plasma enhanced chemical vapor deposition (PECVD) tools, temperature-controlled test heads for die performance testing, temperature-controlled work zones within semiconductor process equipment, thermal shock test bath liquid reservoirs, and constant temperature baths. In some systems, such as etchers, ashers, PECVD chambers, vapor phase soldering devices, and thermal shock testers, the upper desired operating temperature may be as high as 170° C., as high as 200° C., or even as high as 230° C.

Heat can be transferred by placing the heat transfer mechanism in thermal communication with the device. The heat transfer mechanism, when placed in thermal communication with the device, removes heat from the device or provides heat to the device, or maintains the device at a selected temperature or temperature range. The direction of heat flow (from device or to device) is determined by the relative temperature difference between the device and the heat transfer mechanism. The provided apparatus can also include refrigeration systems, cooling systems, testing equipment and machining equipment. In some embodiments, the provided apparatus can be a constant temperature bath or a thermal shock test bath.

In some embodiments, the present disclosure relates to cleaning compositions that include one or more compounds of the present disclosure and one or more co-solvents.

In some embodiments, the compounds of the present disclosure may be present in an amount greater than 50 weight percent, greater than 60 weight percent, greater than 70 weight percent, or greater than 80 weight percent based upon the total weight of the compounds of the present disclosure and the co-solvent(s).

In various embodiments, the cleaning composition may further comprise a surfactant. Suitable surfactants include those surfactants that are sufficiently soluble in the particular hydrofluoroether, and which promote soil removal by dissolving, dispersing or displacing the soil. One useful class of surfactants are those nonionic surfactants that have a hydrophilic-lipophilic balance (HLB) value of less than about 14. Examples include ethoxylated alcohols, ethoxylatedalkyl phenols, ethoxylated fatty acids, alkylarysulfonates, glycerol esters, ethoxylated fluoroalcohols, and fluorinated sulfonamides. Mixtures of surfactants having complementary properties may be used in which one surfactant is added to the cleaning composition to promote oily soil removal and another added to promote water-soluble oil removal. The surfactant, if used, can be added in an amount sufficient to promote soil removal. Typically, surfactant is added in amounts from about 0.1 to 5.0 wt. %, preferably in amounts from about 0.2 to 2.0 wt. % of the cleaning composition.

In illustrative embodiments, the co-solvent may include alcohols, ethers, alkanes, alkenes, haloalkenes, perfluorocarbons, perfluorinated tertiary amines, perfluoroethers, cycloalkanes, esters, ketones, oxiranes, aromatics, haloaromatics, siloxanes, hydrochlorocarbons, hydrochlorofluorocarbons, hydrofluorocarbons, hydrofluoroolefins, hydrochloroolefins, hydrochlorofluoroolefins, or mixtures thereof. Representative examples of co-solvents which can be used in the cleaning composition include methanol, ethanol, isopropanol, t-butyl alcohol, methyl t-butyl ether, methyl t-amyl ether, 1,2-dimethoxyethane, cyclohexane, 2,2,4-trimethylpentane, n-decane, terpenes (e.g., α-pinene, camphene, and limonene), trans-1,2-dichloroethylene, cis-1,2-dichloroethylene, methylcyclopentane, decalin, methyl decanoate, t-butyl acetate, ethyl acetate, diethyl phthalate, 2-butanone, methyl isobutyl ketone, naphthalene, toluene, p-chlorobenzotrifluoride, trifluorotoluene, bis(trifluoromethyl)benzenes, hexamethyl disiloxane, octamethyl trisiloxane, perfluorohexane, perfluoroheptane, perfluorooctane, perfluorotributylamine, perfluoro-N-methyl morpholine, perfluoro-2-butyl oxacyclopentane, methylene chloride, chlorocyclohexane, 1-chlorobutane, 1,1-dichloro-1-fluoroethane, 1,1,1-trifluoro-2,2-dichloroethane, 1,1,1,2,2-pentafluoro-3,3-dichloropropane, 1,1,2,2,3-pentafluoro-1,3-dichloropropane, 2,3-dihydroperfluoropentane, 1,1,1,2,2,4-hexafluorobutane, 1-trifluoromethyl-1,2,2-trifluorocyclobutane, 3-methyl-1,1,2,2-tetrafluorocyclobutane, 1-hydropentadecafluoroheptane, or mixtures thereof.

In some embodiments, the present disclosure relates to a process for cleaning a substrate. The cleaning process can be carried out by contacting a contaminated substrate with a cleaning composition as discussed above. The compounds of the present disclosure can be utilized alone or in admixture with each other or with other commonly-used cleaning solvents, e.g., alcohols, ethers, alkanes, alkenes, haloalkenes, perfluorocarbons, perfluorinated tertiary amines, perfluoroethers, cycloalkanes, esters, ketones, oxiranes, aromatics, haloaromatics, siloxanes, hydrochlorocarbons, hydrochlorofluorocarbons, hydrofluorocarbons, hydrofluoroolefins, hydrochloroolefins, hydrochlorofluoroolefins, or mixtures thereof. Such co-solvents can be chosen to modify or enhance the solvency properties of a cleaning composition for a particular use and can be utilized in ratios (of co-solvent to hydrofluoroolefin compounds) such that the resulting composition has no flash point. If desirable for a particular application, the cleaning composition can further contain one or more dissolved or dispersed gaseous, liquid, or solid additives (for example, carbon dioxide gas, surfactants, stabilizers, antioxidants, or activated carbon).

In some embodiments, the present disclosure relates to cleaning compositions that include one or more compounds of the present disclosure and optionally one or more surfactants. Suitable surfactants include those surfactants that are sufficiently soluble in the compounds of the present disclosure, and which promote soil removal by dissolving, dispersing or displacing the soil. One useful class of surfactants are those nonionic surfactants that have a hydrophilic-lipophilic balance (HLB) value of less than about 14. Examples include ethoxylated alcohols, ethoxylated alkylphenols, ethoxylated fatty acids, alkylaryl sulfonates, glycerol esters, ethoxylated fluoroalcohols, and fluorinated sulfonamides. Mixtures of surfactants having complementary properties may be used in which one surfactant is added to the cleaning composition to promote oily soil removal and another added to promote water-soluble soil removal. The surfactant, if used, can be added in an amount sufficient to promote soil removal. Typically, surfactant may be added in amounts from 0.1 to 5.0 wt. % or from 0.2 to 2.0 wt. % of the cleaning composition.

The cleaning processes of the disclosure can also be used to dissolve or remove most contaminants from the surface of a substrate. For example, materials such as light hydrocarbon contaminants; higher molecular weight hydrocarbon contaminants such as mineral oils and greases; fluorocarbon contaminants such as perfluoropolyethers, bromotrifluoroethylene oligomers (gyroscope fluids), and chlorotrifluoroethylene oligomers (hydraulic fluids, lubricants); silicone oils and greases; solder fluxes; particulates; water; and other contaminants encountered in precision, electronic, metal, and medical device cleaning can be removed.

The cleaning compositions can be used in either the gaseous or the liquid state (or both), and any of known or future techniques for “contacting” a substrate can be utilized. For example, a liquid cleaning composition can be sprayed or brushed onto the substrate, a gaseous cleaning composition can be blown across the substrate, or the substrate can be immersed in either a gaseous or a liquid composition. Elevated temperatures, ultrasonic energy, and/or agitation can be used to facilitate the cleaning. Various different solvent cleaning techniques are described by B. N. Ellis in Cleaning and Contamination of Electronics Components and Assemblies, Electrochemical Publications Limited, Ayr, Scotland, pages 182-94 (1986).

Both organic and inorganic substrates can be cleaned by the processes of the present disclosure. Representative examples of the substrates include metals; ceramics; glass; polycarbonate; polystyrene; acrylonitrile-butadiene-styrene copolymer; natural fibers (and fabrics derived therefrom) such as cotton, silk, fur, suede, leather, linen, and wool; synthetic fibers (and fabrics) such as polyester, rayon, acrylics, nylon, or blends thereof; fabrics comprising a blend of natural and synthetic fibers; and composites of the foregoing materials. In some embodiments, the process may be used in the precision cleaning of electronic components (e.g., circuit boards), optical or magnetic media, or medical devices.

Electrochemical cells (e.g., lithium-ion batteries) are in widespread use worldwide in a vast array of electronic and electric devices ranging from hybrid and electric vehicles to power tools, portable computers, and mobile devices. While generally safe and reliable energy storage devices, lithium-ion batteries are subject to catastrophic failure known as thermal runaway under certain conditions. Thermal runaway is a series of internal exothermic reactions that are triggered by heat. The creation of excessive heat can be from electrical over-charge, thermal over-heat, or from an internal electrical short. Internal shorts are typically caused by manufacturing defects or impurities, dendritic lithium formation and mechanical damage. While there is typically protective circuitry in the charging devices and in the battery packs that will disable the battery in the event of overcharging or overheating, it cannot protect the battery from internal shorts caused by internal defects or mechanical damage.

A thermal management system for electrochemical cell packs (e.g., lithium-ion battery packs) is often required to maximize the cycle life of the cells within the pack. This type of system maintains uniform temperatures of each cell within a pack. High temperatures can increase the capacity fade rate and impedance of lithium-ion batteries while decreasing their lifespan. Ideally, each individual cell within a battery pack will be at the same ambient temperature.

Direct contact fluid immersion of batteries can mitigate low probability, but catastrophic, thermal runaway events while also providing necessary ongoing thermal management for the efficient normal operation of the lithium-ion battery packs. This type of application provides thermal management when the fluid is used with a heat exchange system to maintain a desirable operational temperature range. However, in the event of mechanical damage or an internal short of any of the cells, the fluid would also prevent propagation or cascading of the thermal runaway event to adjacent cells in the pack via evaporative cooling, thus significantly mitigating the risk of a catastrophic thermal runaway event involving multiple cells. Immersion cooling and thermal management of batteries can be achieved using a system designed for single phase or two-phase immersion cooling. In either scenario, the fluids are disposed in thermal communication with the batteries to maintain, increase, or decrease the temperature of the batteries (i.e., heat may be transferred to or from the batteries via the fluid).

In some embodiments, the present disclosure is directed to a thermal management system for an electrochemical cell pack (e.g., a lithium-ion battery pack). The system may include a lithium-ion battery pack and a working fluid in thermal communication with the lithium-ion battery pack. The working fluid may include one or more of the compounds or working fluids of the present disclosure.

The compounds of the present disclosure, alone or in combination, may be employed as fluids for transferring heat from various electronic components (e.g., server computers) by direct contact to provide thermal management and maintain optimal component performance under extreme operation conditions.

In some embodiments, the present disclosure describes the use of the compounds or working fluids of the present disclosure as two-phase immersion cooling fluids for electronic devices, including computer servers. Large scale computer server systems can perform significant workloads and generate a large amount of heat during their operation. A significant portion of the heat is generated by the operation of these servers. Due in part to the large amount of heat generated, these servers are typically rack mounted and air-cooled via internal fans and/or fans attached to the back of the rack or elsewhere within the server ecosystem. As the need for access to greater and greater processing and storage resources continues to expand, the density of server systems (i.e., the amount of processing power and/or storage placed on a single server, the number of servers placed in a single rack, and/or the number of servers and or racks deployed on a single server farm), continue to increase. With the desire for increasing processing or storage density in these server systems, the thermal challenges that result remain a significant obstacle. Conventional air cooling systems (e.g., fan based) require large amounts of power, and the cost of power required to drive such systems increases exponentially with the increase in server densities. Consequently, there exists a need for an efficient, low power usage system for cooling the servers, while allowing for the desired increased processing and/or storage densities of modern server systems.

Two-phase immersion cooling is an emerging cooling technology for the high-performance server computing market which relies on the heat absorbed in the process of vaporizing a liquid (the cooling fluid) to a gas (i.e., the heat of vaporization). The fluids used in this application must meet certain requirements to be viable in the application. For example, the boiling temperature during operation should be in a range between for example 45° C.-75° C. Generally, this range accommodates maintaining the server components at a sufficiently cool temperature while allowing heat to be dissipated efficiently to an ultimate heat sink (e.g., outside air). The fluid must be inert so that it is compatible with the materials of construction and the electrical components. The fluid should be stable such that it does not react with common contaminants such as water or with reagents such as activated carbon or alumina that might be used to scrub the fluid during operation. The global warming potential (GWP, 100 yr ITH) and ozone depletion potential (ODP) of the parent compound and its degradation products should be below acceptable limits, for example, a GWP less than 2000, 1000, 800 or 600 and an ODP less than 0.01, respectively. The compounds of the present disclosure generally meet these requirements.

In another embodiment, the present disclosure describes the use of the compounds or working fluids of the present disclosure as single-phase immersion cooling fluids for electronics. Single phase immersion cooling has a long history in computer server cooling. There is no phase change in single phase immersion. Instead the liquid warms and cools as it flows or is pumped through the computer hardware and a heat exchanger, respectively, thereby transferring heat away from the server. The fluids used in single phase immersion cooling of servers must meet the same requirements as outlined above except that they typically have higher boiling temperatures exceeding about 75 degrees C. to limit evaporative losses.

In some embodiments, the present disclosure may be directed to an immersion cooling system that includes the compounds or working fluids of the present disclosure. Generally, the immersion cooling systems may operate as two-phase vaporization-condensation cooling vessels for cooling one or more heat generating components. In some embodiments, a two-phase immersion cooling system may include a housing having an interior space. Within a lower volume of interior space, a liquid phase of a working fluid having an upper liquid surface (i.e., the topmost level of the liquid phase) may be disposed. The interior space may also include an upper volume extending from the liquid surface up to an upper portion of the housing.

In some embodiments, a heat generating component may be disposed within the interior space such that it is at least partially immersed (and up to fully immersed) in the liquid phase of the working fluid. In some embodiments, the heat generating components may include one or more electronic devices, such as computer servers.

In various embodiments, a heat exchanger (e.g., a condenser) may be disposed within the upper volume. Generally, the heat exchanger may be configured such that it is able to condense a vapor phase of the working fluid that is generated as a result of the heat that is produced by the heat generating element. For example, the heat exchanger may have an external surface that is maintained at a temperature that is lower than the condensation temperature of a vapor phase of the working fluid. In this regard, at the heat exchanger, a rising vapor phase of the working fluid may be condensed back to liquid phase or condensate by releasing latent heat to the heat exchanger as the rising vapor phase comes into contact with the heat exchanger. The resulting condensate may then be returned to the liquid phase disposed in the lower volume.

In some embodiments, the present disclosure may be directed to an immersion cooling system which operates by single-phase immersion cooling. Generally, the single phase immersion cooling system is similar to that of the two-phase system in that it may include a heat generating component disposed within the interior space of a housing such that it is at least partially immersed (and up to fully immersed) in the liquid phase of the working fluid. The single-phase system may further include a pump and a heat exchanger, the pump operating to move the working fluid to and from the heat generating components and the heat exchanger, and the heat exchanger operating to cool the working fluid. The heat exchanger may be disposed within or external to the housing.

In some embodiments, the present disclosure may be directed to methods for cooling electronic components. Generally, the methods may include at least partially immersing a heat electronic generating component (e.g., a computer server) in a liquid that includes the compounds or working fluids of the present disclosure. The method may further include transferring heat from the heat generating electronic component using the compounds or working fluids of the present disclosure.

In some embodiments, the present disclosure is directed to a fire extinguishing composition. The composition may include one or more compounds of the present disclosure and one or more co-extinguishing agents.

In illustrative embodiments, the co-extinguishing agent may include hydrofluorocarbons, hydrochlorofluorocarbons, perfluorocarbons, perfluoropolyethers, hydrofluoroethers, hydrofluoropolyethers, chlorofluorocarbons, bromofluorocarbons, bromochlorofluorocarbons, hydrobromocarbons, iodofluorocarbons, fluorinated ketones, hydrofluorocarbons, hydrochlorofluorocarbons, perfluorocarbons, perfluoropolyethers, hydrofluoroethers, hydrofluoropolyethers, chlorofluorocarbons, bromofluorocarbons, bromochlorofluorocarbons, iodofluorocarbons, hydrobromofluorocarbons, fluorinated ketones, hydrobromocarbons, fluorinated olefins, hydrofluoroolefins, fluorinated sulfones, fluorinated vinylethers, unsaturated fluoro-ethers, bromofluoroolefins, chlorofluorolefins, iodofluoroolefins, fluorinated vinyl amines, fluorinated aminopropenes and mixtures thereof.

Such co-extinguishing agents can be chosen to enhance the extinguishing capabilities or modify the physical properties (e.g., modify the rate of introduction by serving as a propellant) of an extinguishing composition for a particular type (or size or location) of fire and can preferably be utilized in ratios (of co-extinguishing agent to hydrofluoroolefin compound) such that the resulting composition does not form flammable mixtures in air.

In some embodiments, the compounds of the present disclosure and the co-extinguishing agent may be present in the fire extinguishing composition in amounts sufficient to suppress or extinguish a fire. The compounds of the present disclosure and the co-extinguishing agent can be in a weight ratio of from about 9:1 to about 1:9.

### Examples

Objects and advantages of this disclosure are further illustrated by the following illustrative examples. Unless otherwise noted, all parts, percentages, ratios, etc. in the examples and the rest of the specification are by weight, and all reagents used in the examples were obtained, or are available, from general chemical suppliers such as, for example, Sigma-Aldrich Corp., Saint Louis, Mo., US or may be synthesized by conventional methods.

The following abbreviations are used herein: mL=milliliters, L=liters, mol=moles, mmol=millimoles, min=minutes, h or hr=hours, sec=seconds, g=grams, ° C.=degrees Celsius, mp=melting point, cSt=centiStokes. “RT” or “room temperature” refers to an ambient temperature of approximately 20-25° C., with an average of 23° C.

TABLE 1
Materials

| Material | Description | Source |
|---|---|---|
| CsF | Cesium fluoride | Alfa Aesar, Haverhill, |
| | | MA, US |
| KF | Potassium fluoride | Sigma-Aldrich Corp., |
| | | Saint Louis, MO, US |
| 18-Crown-6 | 1,4,7,10,13,16- | Oakwood Products, Inc., |
| | hexaoxacyclooctadecane | Augusta, GA, US |
| TFE | Tetrafluoroethylene | 3M Company, |
| | | Maplewood, MN, USA |
| α-Pinene | α-Pinene | Sigma-Aldrich Corp., |
| | | Saint Louis, MO, US |
| Perfluoropropionyl | 2,2,3,3,3-Pentafluoropropionyl | 3M Company, |
| fluoride | fluoride | Maplewood, MN, USA |
| Hexafluoroacetone | 1,1,1,3,3,3-hexafluoropropan-2-one | SynQuest Laboratories, |
| | | Alachua FL, US |
| TMSCF₃ | Trifluoromethyltrimethylsilane | Oakwood Products, Inc., |
| | | Augusta, GA, US |
| TMSCF₂CF₃ | (Pentafluoroethyl) trimethylsilane | Oakwood Products, Inc., |
| | | Augusta, GA, US |
| Me—I | Iodomethane | Alfa Aesar, Haverhill, |
| | | MA, US |
| Et—I | Iodoethane | Sigma-Aldrich Corp., |
| | | Saint Louis, MO, US |
| DMS | Dimethyl sulfate | Oakwood Products, Inc., |
| | | Augusta, GA, US |
| DMF | N,N-Dimethylformamide | Sigma-Aldrich Corp., |
| | | Saint Louis, MO, US |
| 50% NH₄OH | 50% Ammonium hydroxide | Alfa Aesar, Haverhill, |
| | | MA, US |
| TEA | Triethylamine | Alfa Aesar, Haverhill, |
| | | MA, US |
| DABCO | 1,4-diazabicyclo[2.2.2]octane | Alfa Aesar, Haverhill, |
| | | MA, US |
| TMEDA | N,N,N,N-Tetramethylethylene | Alfa Aesar, Haverhill, |
| | diamine | MA, US |
| NMP | N-Methylpyrrolidinone | Sigma-Aldrich Corp., |
| | | Saint Louis, MO, US |
| Na₂CO₃ | Sodium carbonate | Sigma-Aldrich Corp., |
| | | Saint Louis, MO, US |
| 2,2,3,3-tetrafluoro-3- | 2,2,3,3-tetrafluoro-3- | Prepared as described in |
| (perfluoromorpholino) | (perfluoromorpholino) propanoyl | Abe, T. et al, |
| propanoyl fluoride | fluoride | J. Fluorine Chem., |
| | | (2001), 111, 115-128. |
| 2,2-difluoro-2- | 2,2-difluoro-2-(2,2,3,3,5,5,6,6- | Prepared as described in |
| (2,2,3,3,5,5,6,6- | octafluoromorpholin-4-yl)acetyl | Abe, T. et al, |
| octafluoromorpholin-4- | fluoride | J. Fluorine Chem., |
| yl)acetyl fluoride | | (2001), 111, 115-128. |
| 2,2,3,3-tetrafluoro-3- | 2,2,3,3-tetrafluoro-3- | SynQuest Laboratories, |
| (trifluoromethoxy) | (trifluoromethoxy) propanoyl fluoride | Alachua FL, US |
| propanoyl fluoride | | |
| 2,2,3,3,4,4-hexafluoro- | 2,2,3,3,4,4-hexafluoro-4- | Prepared as described in |
| 4-(perfluoroethoxy) | (perfluoroethoxy) butanoyl fluoride | Japanese Patent |
| butanoyl fluoride | | Application |
| | | JP2005225816A |
| CF₃CF₂CF₂CH₂OTf | 2,2,3,3,4,4,4-Heptafluorobutyl | Oakwood Products, Inc., |
| | trifluoromethanesulfonate | Augusta, GA, US |
| CF₃CH₂OTf | 2,2,2-Trifluoroethyl trifluoromethane | Oakwood Products, Inc., |
| | sulfonate | Augusta, GA, US |
| CF₃CH₂ONf | 2,2,2-Trifluoroethyl nonafluorobutane | SynQuest Laboratories, |
| | sulfonate | Alachua FL, US |
| CF₃CF₂CH₂OTf | 2,2,3,3,3-Pentafluoropropyl | Oakwood Products, Inc., |
| | trifluoromethane sulfonate | Augusta, GA, US |
| CF₃CF₂CH₂ONf | 2,2,3,3,3-Pentafluoropropyl | SynQuest Laboratories, |
| | nonafluorobutane sulfonate | Alachua FL, US |
| CF₃CF₂CF₂CH₂ONf | 2,2,3,3,4,4,4-Heptafluorobutyl | SynQuest Laboratories, |
| | nonafluoro-butane sulfonate | Alachua FL, US |
| Diglyme | Diethylene glycol dimethyl ether | Sigma-Aldrich Corp., |
| | | Saint Louis, MO, US |
| Tetraglyme | Tetraethylene glycol dimethyl ether | Alfa Aesar, Haverhill, |
| | | MA, US |
| FC-770 | FLUORINERT FC-770 Fluorinated | 3M Company, |
| | Liquid | Maplewood, MN, USA |
| Comparative Example | FLUORINERT FC-72 Fluorinated | 3M Company, |
| CE1 | Liquid | Maplewood, MN, USA |
| Comparative Example | FLUORINERT FC-77 Fluorinated | 3M Company, |
| CE2 | Liquid | Maplewood, MN, USA |
| Comparative Example | NOVEC 7500 Engineered Fluid | 3M Company, |
| CE3 | | Maplewood, MN, USA |
| Comparative Example | 1,1,1,2,3,4,4,4-octafluoro-2-methoxy- | Prepared as described in |
| CE4 | 3-(trifluoromethyl)butane | U.S. Pat. No. 5,750,797 |
| Comparative Example | 2-(difluoro(methoxy)methyl)- | Prepared as described in |
| CE5 | 1,1,1,2,3,3,4,4,4-nonafluorobutane | U.S. Pat. No. 5,750,797 |
| Comparative Example | OPTEON SF-10 Fluorinated Fluid | Chemours, Wilmington, |
| CE6 | | DE, US |
| C-9 hydrocarbon | C₉H₂₀, mp = −53.5° C. | Sigma-Aldrich Corp., |
| | | Saint Louis, MO, US |
| C-10 hydrocarbon | C₁₀H₂₂, mp = −29.7° C. | Sigma-Aldrich Corp., |
| | | Saint Louis, MO, US |
| C-11 hydrocarbon | C₁₂H₂₄, mp = −25° C. | Sigma-Aldrich Corp., |
| | | Saint Louis, MO, US |
| C-12 hydrocarbon | C₁₂H₂₆, mp = −9.6° C. | Sigma-Aldrich Corp., |
| | | Saint Louis, MO, US |
| C-13 hydrocarbon | C₁₃H₂₈, mp = −5.4° C. | Sigma-Aldrich Corp., |
| | | Saint Louis, MO, US |
| C-14 hydrocarbon | C₁₄H₃₀, mp = 5.9° C. | Sigma-Aldrich Corp., |
| | | Saint Louis, MO, US |

### Sample Preparation

### Example 1: 1,1,1,2,2,4,4,4-octafluoro-3-methoxy-3-(trifluoromethyl)butane)

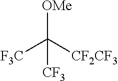

Method A: A 4 L (7.42 L net volume) stainless steel kettle was charged with diglyme (994 g, 7.4 mol; contained according to titration in sense of Karl Fischer 70 ppm water), cesium fluoride (184 g, 1.2 mol), and α-pinene (1 g, 0.01 mol). Within 114 h under stirring with 400 rpm, TFE (180 g, 1.8 mol) and hexafluoroacetone (206 g, 1.2 mol) were reacted at 90° C. until a constant pressure plateau (8.1 bar→1.8 bar) was observed. The temperature was then allowed to cool to room temperature and the resultant stirring mixture was sparged with N₂ gas to remove any excess TFE. The reactor was then drained to afford a solution of Cs[(CF₃)₂(C₂F₅)CO] (356 g, 0.85 mol, 71% yield) in diglyme, which was confirmed by ¹⁹F NMR and by GC-MS via acidification of the cesium salt with H₂SO₄. The Cs[(CF₃)₂(C₂F₅)CO] was used in the next step without further purification.

To a 3-neck flask equipped with a magnetic stir bar and reflux condenser were charged 1,1,1,3,3,4,4,4-octafluoro-2-(trifluoromethyl)butan-2-olate cesium salt (300 g of a 33 wt % solution in diglyme, 237 mmol). The resultant reaction mixture was heated to 70° C. followed by the dropwise addition of iodomethane (17 mL, 273 mmol). The reaction mixture was allowed to stir overnight at the same temperature. After cooling to room temperature, H₂O (400 mL) was added and the mixture was transferred to a 2 L separatory funnel. The bottom, fluorochemical layer was then separated and analyzed by GC-FID indicating a 58% yield of the title compound. Distillation afforded the desired 1,1,1,2,2,4,4,4-octafluoro-3-methoxy-3-(trifluoromethyl)butane (77° C., 740 mm/Hg, 32.1 g, 45% isolated yield) as a colorless liquid. The identity of the purified composition was confirmed by GC-MS analysis. A toxicity screening study in rats indicated that the 4-hour inhalation LC50 of this compound is >1,840 ppm.

Method B: A 600 mL stainless steel reaction vessel was charged with tetraglyme (120 mL), potassium fluoride (24.1 g, 415 mmol), and 18-crown-6 (15 g, 57 mmol). The sealed reaction vessel was then evacuated under reduced pressure followed by the addition of 2,2,3,3,3-pentafluoropropionyl fluoride (65 g, 390 mmol). To the stirring mixture was then added trifluoromethyltrimethylsilane (114 g, 803 mmol) over the course of 1 h at a rate which avoided reaction temperatures rising above 45° C. After complete addition, the resultant reaction mixture was allowed to stir overnight without heating. The reaction mixture was then transferred to a 500 mL 3-neck round-bottom flask equipped with a stir bar, reflux condenser, and temperature probe. With stirring, dimethyl sulfate (49.4 g, 392 mmol) was slowly added to the heated (45° C.) mixture. 15 minutes following complete addition, salt formation was observed. The mixture was allowed to stir overnight at the same temperature. The resultant reaction mixture was then allowed to cool to room temperature followed by the addition of H₂O (100 mL) and ammonium hydroxide (100 mL of a 50% solution in H₂O). The contents were transferred to a 1 L separatory funnel and the fluorochemical layer was collected and analyzed by GC-FID indicating formation of the desired 1,1,1,2,2,4,4,4-octafluoro-3-methoxy-3-(trifluoromethyl)butane (60% GC-FID yield). The identity of the desired composition in the fluorochemical layer was confirmed by GC-MS analysis.

### Example 2: 2-ethoxy-1,1,1,3,3,4,4,4-octafluoro-2-(trifluoromethyl)butane

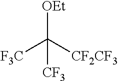

A 600 mL stainless steel reaction vessel was charged with N,N-dimethylformamide (100 mL), potassium fluoride (15.4 g, 265 mmol), and 18-crown-6 (12.7 g, 48 mmol). The sealed reaction vessel was then evacuated under reduced pressure followed by the addition of 2,2,3,3,3-pentafluoropropionyl fluoride (40.2 g, 242 mmol). To the stirring mixture was then added trifluoromethyltrimethylsilane (72.2 g, 508 mmol) over the course of 1 h at a rate which avoided reaction temperatures rising above 45° C. After complete addition, the resultant reaction mixture was allowed to stir overnight without heating. The reaction mixture was then transferred to a 250 mL 3-neck round-bottom flask equipped with a stir bar, reflux condenser, and temperature probe. With heating (45° C.), iodoethane (41.3 g, 265 mmol) was slowly added to the reaction mixture over the course of 30 min.

Approximately 15 min after complete addition of iodomethane, salt formation was observed. The reaction mixture was allowed to stir overnight at the same temperature. The mixture was allowed to cool to room temperature followed by the addition of water (200 mL) and was then transferred to a 1 L separatory funnel. The fluorochemical phase was collected and analyzed by GC-FID indicating formation of the desired 2-ethoxy-1,1,1,3,3,4,4,4-octafluoro-2-(trifluoromethyl)butane (54% GC-FID yield). Concentric tube distillation (91° C., 740 mm/Hg) afforded the desired 2-ethoxy-1,1,1,3,3,4,4,4-octafluoro-2-(trifluoromethyl)butane (39 g, 51% isolated yield). The identity of the isolated composition was confirmed by GC-MS analysis.

### Example 3: 1,1,1,2,2,4,4,5,5,5-decafluoro-3-methoxy-3-(1,1,2,2,2-pentafluoroethyl)pentane

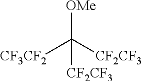

A 4 L (7.42 L net volume) stainless steel kettle was charged with diglyme (1001 g, 7.5 mol; contained according to titration in sense of Karl Fischer 70 ppm water), CsF (229 g, 1.5 mol), and α-pinene (1 g, 0.01 mol). Within 125 h under stirring with 400 rpm, TFE (330 g, 3.3 mol) and 2,2,3,3,3-pentafluoropropionyl fluoride (250 g, 1.5 mol) were reacted at 90° C. until a constant pressure plateau (7.1 bar→4.4 bar) was observed. The temperature was then allowed to cool to room temperature and the resultant stirring mixture was sparged with N₂ gas to remove any excess TFE. The reactor was then drained to afford a solution of Cs[(C₂F₅)₃CO] (241 g, 0.47 mol) was obtained in 31% yield confirmed by ¹⁹F NMR and by GC-MS via acidification of the cesium salt with H₂SO₄. The Cs[(C₂F₅)₃CO] was used in the next step without further purification.

To a round bottom 3-neck flask equipped with a magnetic stir bar, reflux condenser, and temperature probe were charged cesium 1,1,1,2,2,4,4,5,5,5-decafluoro-3-(1,1,2,2,2-pentafluoroethyl)pentan-3-olate (400 g of a 31 wt % solution in diglyme, 239 mmol) and sodium carbonate (24.3 g, 229 mmol). The resultant mixture was heated to 70° C. followed by the dropwise addition of iodomethane (40.8 g, 287 mmol). After an overnight stir at the same temperature, the reaction mixture was allowed to cool to room temperature followed by the addition of H₂O (400 mL). The contents were then transferred to a 2 L separatory funnel and the fluorochemical layer was collected and analyzed by GC-FID indicating formation of the desired 1,1,1,2,2,4,4,5,5,5-decafluoro-3-methoxy-3-(1,1,2,2,2-pentafluoroethyl)pentane (59% GC-FID yield). Concentric tube distillation (126° C., 740 mm/Hg, 32.1 g, 34% isolated yield) afforded the desired 1,1,1,2,2,4,4,5,5,5-decafluoro-3-methoxy-3-(1,1,2,2,2-pentafluoroethyl)pentane. The identity of the isolated composition was confirmed by GC-MS analysis.

### Example 4: 3-ethoxy-1,1,1,2,2,4,4,5,5,5-decafluoro-3-(1,1,2,2,2-pentafluoroethyl)pentane

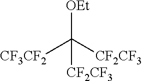

A 4 L (7.42 L net volume) stainless steel kettle was charged with diglyme (1001 g, 7.5 mol; contained according to titration in sense of Karl Fischer 70 ppm water), CsF (229 g, 1.5 mol), and α-pinene (1 g, 0.01 mol). Within 125 h under stirring with 400 rpm, TFE (330 g, 3.3 mol) and 2,2,3,3,3-pentafluoropropionyl fluoride (250 g, 1.5 mol) were reacted at 90° C. until a constant pressure plateau (7.1 bar→4.4 bar) was observed. The temperature was then allowed to cool to room temperature and the resultant stirring mixture was sparged with N₂ gas to remove any excess TFE. The reactor was then drained to afford a solution of Cs[(C₂F₅)₃CO] (241 g, 0.47 mol) was obtained in 31% yield confirmed by ¹⁹F NMR and by GC-MS via acidification of the cesium salt with H₂SO₄. The Cs[(C₂F₅)₃CO] was used in the next step without further purification.

To a round bottom 3-neck flask equipped with a magnetic stir bar, reflux condenser, and temperature probe were charged cesium 1,1,1,2,2,4,4,5,5,5-decafluoro-3-(1,1,2,2,2-pentafluoroethyl)pentan-3-olate (200 g of a 33 wt % solution in diglyme, 127 mmol) and sodium carbonate (5.4 g, 51 mmol). The resultant mixture was heated to 70° C. followed by the dropwise addition of iodoethane (21.9 g, 140 mmol). After an overnight stir at the same temperature, the reaction mixture was allowed to cool to room temperature followed by the addition of H₂O (150 mL). The contents were transferred to a 1 L separatory funnel and the fluorochemical layer was collected and purified via concentric tube distillation (138° C., 740 mm/Hg) affording the desired 3-ethoxy-1,1,1,2,2,4,4,5,5,5-decafluoro-3-(1,1,2,2,2-pentafluoroethyl)pentane (25.6 g, 49% isolated yield) as a colorless liquid.

### Example 5: 1,1,1,2,2,3,3,4,4,6,6,6-dodecafluoro-5-methoxy-5-(trifluoromethyl)hexane

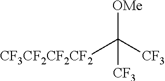

To a round bottom flask equipped with a magnetic stir bar, Claisen head adapter, and a reflux condenser were added 18-crown-6 (3.0 g, 11.3 mmol), potassium fluoride (3.9 g, 67.7 mmol), and N,N-dimethylformamide (20 mL). To the resultant stirring mixture was then added 2,2,3,3,4,4,5,5,5-nonafluoropentanoyl fluoride (15 g, 56 mmol) slowly. The temperature was then slowly raised (50° C.) followed by the slow addition of trifluoromethyltrimethylsilane (17.5 g, 123 mmol) over the course of 20 min. The mixture was then stirred at the same temperature overnight before the addition of iodomethane (8.8 g, 62 mmol) followed by an additional overnight stir at the same temperature. The reaction was then allowed to cool to room temperature followed by the addition of H₂O (50 mL). The contents were transferred to a 500 mL separatory funnel and the fluorochemical phase was collected and analyzed by GC-FID indicating formation of the desired 1,1,1,2,2,3,3,4,4,6,6,6-dodecafluoro-5-methoxy-5-(trifluoromethyl)hexane (55% GC-FID yield). Concentric tube distillation (126° C., 740 mm/Hg) afforded the desired 1,1,1,2,2,3,3,4,4,6,6,6-dodecafluoro-5-methoxy-5-(trifluoromethyl)hexane (6.7 g, 30% yield) as a colorless liquid. The identity of the desired composition was confirmed by GC-MS analysis.

### Example 6: 1,1,2,2,3,3,4,4-octafluoro-5-methoxy-5-(trifluoromethyl)cyclopentane

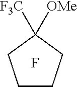

To a 3-neck round bottom flask equipped with a magnetic stir bar, reflux condenser, and temperature probe was charged potassium fluoride (8.1 g, 140 mmol) and 18-crown-6 (2.4 g, 9.1 mmol). The flask was evacuated and backfilled with N₂ three times. The vessel was then charged with N,N-dimethylformamide (30 mL). To the resultant stirring mixture was slowly added 2,2,3,3,4,4,5,5-octafluorocyclopentanone (25.1 g, 110 mmol) followed by the dropwise addition of trifluoromethyltrimethylsilane (17.6 g, 124 mmol) over the course of 30 min. The reaction mixture was allowed to stir overnight at room temperature before the reaction temperature was raised (50° C.) followed by the addition of iodomethane (17.1 g, 121 mmol) and an overnight stir at the same temperature. The resultant reaction mixture was allowed to cool to room temperature before the addition of H₂O (100 mL). The contents were transferred to a 250 mL separatory funnel and the fluorochemical layer was collected and analyzed by GC-FID indicating formation of the desired 1,1,2,2,3,3,4,4-octafluoro-5-methoxy-5-(trifluoromethyl)cyclopentane (9% GC-FID yield). The identity of the desired composition in the fluorochemical layer was confirmed by GC-MS analysis.

### Example 7: 1,1,1,2,4,5,5,5-nonafluoro-3-methoxy-3,4-bis(trifluoromethyl)pentane

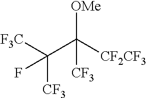

To a 3-neck round bottom flask equipped with a reflux condenser, temperature probe, and magnetic stir bar were added potassium fluoride (6.07 g, 104 mmol) and 18-crown-6 (5.02 g, 19.0 mmol). The flask was evacuated and backfilled with N₂ three times followed by the addition of N,N-dimethylformamide (40 mL). To the resultant stirring mixture was slowly added 1,1,1,2,2,4,5,5,5-nonafluoro-4-(trifluoromethyl)pentan-3-one (30.2 g, 96 mmol) followed by the addition of trifluoromethyltrimethylsilane (14.9 g, 105 mmol) at a rate which avoided internal temperature rises above 32° C. The resultant mixture was allowed to stir at room temperature overnight followed by the dropwise addition of iodomethane (14.9 g, 105 mmol). After an overnight stir at 50° C., the resultant reaction mixture was allowed to cool to room temperature before the addition of H₂O (100 mL). The contents were transferred to a 250 mL separatory funnel. The fluorochemical layer was collected and analyzed by GC-FID indicating formation of the desired 1,1,1,2,2,4,5,5,5-nonafluoro-3-methoxy-3,4-bis(trifluoromethyl)pentane (46% GC-FID yield). The identity of the desired composition in the fluorochemical layer was confirmed by GC-MS analysis.

### Example 8: 1,1,2,2,3,3,5,5,5-nonafluoro-4-methoxy-N,N-bis(perfluoropropyl)-4-(trifluoromethyl)pentan-1-amine

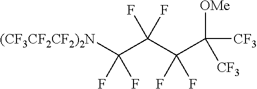

A 3-neck round bottom flask equipped with a reflux condenser, temperature probe, and magnetic stir bar was charged with tetraglyme (75 mL) and cesium fluoride (15.2 g, 100 mmol). To the resultant stirring mixture was slowly added 4-(bis(perfluoropropyl)amino)-2,2,3,3,4,4-hexafluorobutanoyl fluoride (50.1 g, 91.2 mmol) followed by the dropwise addition of trifluoromethyltrimethylsilane (27.2 g, 191 mmol) at a rate which avoided a reaction temperature rise above 30° C. After an overnight stir, dimethyl sulfate (11.5 g, 91.2 mmol) was added dropwise to avoid internal temperature spikes above 30 C. The resultant mixture was then heated (45° C.) followed by an overnight stir at the same temperature. The resultant reaction mixture was then allowed to cool to room temperature followed by the addition of ammonium hydroxide (100 mL of a saturated solution in water). Removal of the aqueous layer afforded a crude fluorochemical layer for which GC-FID analysis indicated a 55% of the desired 1,1,2,2,3,3,5,5,5-nonafluoro-4-methoxy-N,N-bis(perfluoropropyl)-4-(trifluoromethyl)pentan-1-amine. Distillation of the crude fluorochemical material (113° C., 20 mm/Hg) afforded the desired 1,1,2,2,3,3,5,5,5-nonafluoro-4-methoxy-N,N-bis(perfluoropropyl)-4-(trifluoromethyl)pentan-1-amine (24.9 g, 40% isolated yield) as a colorless liquid. The identity of the isolated material was confirmed by GC-MS analysis.

### Example 9: 2,2,3,3,5,5,6,6-octafluoro-4-(1,1,2,2,4,4,5,5,5-nonafluoro-3-methoxy-3-(perfluoroethyl)pentyl)morpholine

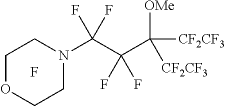

A 3-neck round bottom flask equipped with a reflux condenser, temperature probe, and magnetic stir bar was charged with tetraglyme (40 mL) and cesium fluoride (12.1 g, 79.7 mmol). To the resultant stirring mixture was slowly added 2,2,3,3-tetrafluoro-3-(perfluoromorpholino)propanoyl fluoride (25 g, 66 mmol) followed by the dropwise addition of (pentafluoroethyl)trimethylsilane (26.0 g, 133 mmol) at a rate which avoided a reaction temperature rise above 30° C. After an overnight stir, dimethyl sulfate (8.4 g, 66 mmol) was added dropwise to avoid internal temperature spikes above 30 C. The resultant mixture was then heated (45° C.) followed by an overnight stir at the same temperature. The resultant reaction mixture was then allowed to cool to room temperature followed by the addition of ammonium hydroxide (75 mL of a saturated solution in water). Removal of the aqueous layer afforded a crude fluorochemical layer for which GC-FID analysis indicated a 58% yield of the desired 2,2,3,3,5,5,6,6-octafluoro-4-(1,1,2,2,4,4,5,5,5-nonafluoro-3-methoxy-3-(perfluoroethyl)pentyl)morpholine. Distillation of the crude fluorochemical material (95° C., 20 mm/Hg) afforded the desired 2,2,3,3,5,5,6,6-octafluoro-4-(1,1,2,2,4,4,5,5,5-nonafluoro-3-methoxy-3-(perfluoroethyl)pentyl)morpholine (15.2 g, 38% isolated yield) as a colorless liquid. The identity of the isolated material was confirmed by GC-MS analysis.

### Example 10: 2,2,3,3,5,5,6,6-octafluoro-4-(1,1,3,3,3-pentafluoro-2-methoxy-2-(trifluoromethyl)propyl)morpholine

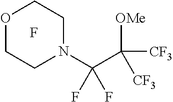

A round bottom flask equipped with a magnetic stir bar, Claisen head adapter, and a reflux condenser was charged with 18-crown-6 (1.9 g, 7.0 mmol), potassium fluoride (2.45 g, 42.2 mmol), and tetraglyme (20 mL). The resultant mixture was then stirred and slowly charged with 2,2-difluoro-2-(2,2,3,3,5,5,6,6-octafluoromorpholin-4-yl)acetyl fluoride (11.5 g, 35.2 mmol). To the heated (50° C.) mixture was then slowly added trifluoromethyltrimethylsilane (11.3 g, 79.5 mmol) over the course of 20 minutes followed by an overnight stir at the same temperature. To the resultant reaction mixture was then added iodomethane (2.5 mL, 40 mmol) at the same temperature followed by a 3 hour stir. The mixture was then allowed to cool to room temperature followed by the addition of water (50 mL). Removal of the aqueous layer afforded a crude fluorochemical layer for which GC-FID analysis indicated a 62% yield of the desired 2,2,3,3,5,5,6,6-octafluoro-4-(1,1,3,3,3-pentafluoro-2-methoxy-2-(trifluoromethyl)propyl)morpholine. The identity of the desired material was confirmed by GC-MS analysis.

### Example 11: 1,1,1,3,3,4,4-heptafluoro-2-methoxy-4-(trifluoromethoxy)-2-(trifluoromethyl)butane

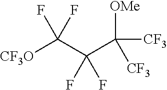

A round bottom flask equipped with a magnetic stir bar, Claisen head adapter, and a dry ice reflux condenser was charged with 18-crown-6 (9.1 g, 34 mmol), potassium fluoride (13.0 g, 224 mmol), and DMF (75 mL). The resultant mixture was then stirred and slowly charged with 2,2,3,3-tetrafluoro-3-(trifluoromethoxy)propanoyl fluoride (40.1 g, 173 mmol) via a plastic line and at a rate which avoided temperature spikes above 30° C. To the heated resultant reaction mixture was then slowly added trifluoromethyltrimethylsilane (51.5 g, 362 mmol) over the course of 20 minutes to avoid temperature increases greater than 40° C. After complete addition, the reaction mixture was allowed to stir overnight at room temperature. To the resultant reaction mixture was then added iodomethane (25.8 g, 181 mmol) followed by a 3 h stir at 40° C. The mixture was then allowed to cool to room temperature followed by the addition of water (100 mL). Removal of the aqueous layer afforded a crude fluorochemical layer for which GC-FID analysis indicated a 55% yield of the desired 1,1,1,3,3,4,4-heptafluoro-2-methoxy-4-(trifluoromethoxy)-2-(trifluoromethyl)butane. Distillation of the crude fluorochemical material (103.6° C., 740 mm/Hg) afforded the desired 1,1,1,3,3,4,4-heptafluoro-2-methoxy-4-(trifluoromethoxy)-2-(trifluoromethyl)butane (20.8 g, 33% isolated yield) as a colorless liquid. The identity of the isolated material was confirmed by GC-MS analysis.

### Example 12: 1,1,1,3,3,4,4,5,5-nonafluoro-2-methoxy-5-(perfluoroethoxy)-2-(trifluoromethyl)pentane

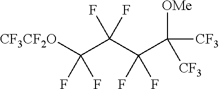

A 3-neck round bottom flask equipped with a magnetic stir bar, and a reflux condenser, and temperature probe was charged with 18-crown-6 (4.8 g, 18 mmol), potassium fluoride (6.3 g, 110 mmol), and DMF (40 mL). The flask was then evacuated and back-filled with N₂ three times. 2,2,3,3,4,4-Hexafluoro-4-(perfluoroethoxy)butanoyl fluoride (30.0 g, 90.4 mmol) was then slowly added to the stirring mixture at a rate which avoided temperature spikes above 30° C. To the mixture was then slowly added trifluoromethyltrimethylsilane (27.0 g, 190 mmol) over the course of 20 minutes to avoid temperature increases greater than 40° C. After complete addition, the reaction mixture was allowed to stir overnight at room temperature. To the resultant reaction mixture was then added dimethyl sulfate (11.4 g, 90.4 mmol) followed by a 3 hour stir at 40° C. The mixture was then allowed to cool to room temperature followed by the addition of saturated ammonium hydroxide (50 mL). Removal of the aqueous layer afforded a crude fluorochemical layer for which GC-FID analysis indicated a 61.6% yield of the desired 1,1,1,3,3,4,4,5,5-nonafluoro-2-methoxy-5-(perfluoroethoxy)-2-(trifluoromethyl)pentane. Distillation of the crude fluorochemical material (150.7° C., 740 mm/Hg) afforded the desired 1,1,1,3,3,4,4,5,5-nonafluoro-2-methoxy-5-(perfluoroethoxy)-2-(trifluoromethyl)pentane (25 g, 58% isolated yield) as a colorless liquid. The identity of the isolated material was confirmed by GC-MS analysis.

### Example 13: 1,1,1,3,3,4,4,5,5-nonafluoro-2-methoxy-5-(perfluoropropoxy)-2-(trifluoromethyl)pentane

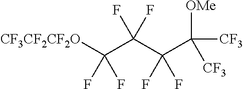

A 3-neck round bottom flask equipped with a magnetic stir bar, reflux condenser, and temperature probe was charged with cesium fluoride (21.0 g, 138 mmol), and diglyme (40 mL). 2,2,3,3,4,4-hexafluoro-4-(perfluoropropoxy)butanoyl fluoride (36 g, 94 mmol) was then slowly added to the stirring mixture at a rate which avoided temperature spikes above 30° C. To the mixture was then slowly added trifluoromethyltrimethylsilane (28.1 g, 198 mmol) over the course of 20 minutes to avoid temperature increases greater than 30° C. After complete addition, the reaction mixture was allowed to stir overnight at room temperature. To the resultant reaction mixture was then added dimethyl sulfate (11.9 g, 94.3 mmol) followed by a 3 h stir at 30° C. After the resultant mixture was allowed to cool to room temperature, saturated ammonium hydroxide (50 mL) was added. Separation of the aqueous afforded a crude fluorochemical layer for which GC-FID analysis indicated a 49% yield of the desired 1,1,1,3,3,4,4,5,5-nonafluoro-2-methoxy-5-(perfluoropropoxy)-2-(trifluoromethyl)pentane. Distillation of the crude fluorochemical material (167° C., 740 mm/Hg) afforded the desired 1,1,1,3,3,4,4,5,5-nonafluoro-2-methoxy-5-(perfluoropropoxy)-2-(trifluoromethyl)pentane (19.9 g, 41% isolated yield) as a colorless liquid. The identity of the isolated material was confirmed by GC-MS analysis.

### Example 14: 1,1,1,2,2,4,4,5,5,5-decafluoro-3-methoxy-3-(1,1,2,2-tetrafluoro-2-(trifluoromethoxy)ethyl)pentane

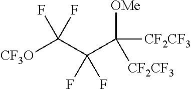

A 3-neck round bottom flask equipped with a magnetic stir bar, reflux condenser, and temperature probe was charged with cesium fluoride (10.8 g, 71.1 mmol), and tetraglyme (30 mL). 2,2,3,3-Tetrafluoro-3-(trifluoromethoxy)propanoyl fluoride (15 g, 65 mmol) was then slowly added to the stirring mixture at a rate which avoided temperature spikes above 30° C. To the mixture was then slowly added (pentafluoroethyl)trimethylsilane (24.9 g, 130 mmol) over the course of 20 minutes to avoid temperature increases greater than 30° C. After complete addition, the reaction mixture was allowed to stir overnight at room temperature. To the resultant reaction mixture was then added dimethyl sulfate (8.2 g, 65 mmol) followed by a 3 h stir at 30° C. The resultant reaction mixture was then allowed to cool to room temperature followed by the addition of saturated ammonium hydroxide (50 mL). The aqueous was removed and distillation of the crude fluorochemical layer (148° C., 740 mm/Hg) afforded the desired 1,1,1,2,2,4,4,5,5,5-decafluoro-3-methoxy-3-(1,1,2,2-tetrafluoro-2-(trifluoromethoxy)ethyl)pentane (16.2 g, 54% isolated yield) as a colorless liquid. The identity of the isolated material was confirmed by GC-MS analysis.

### Example 15: 1,1,1,2,2,4,4,4-octafluoro-3-(2,2,2-trifluoroethoxy)-3-(trifluoromethyl)butane

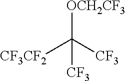

Step 1: To a 600 mL stainless steel reaction vessel equipped with an overhead stirrer were added tetraglyme (100 mL), potassium fluoride (16.1 g, 277 mmol), and 18-Crown-6 (10.5 g, 39.7 mmol). The reaction vessel was sealed and evacuated. To the vessel was then added 2,2,3,3,3-pentafluoropropanoyl fluoride (43.0 g, 259 mmol) in one portion. To the stirring mixture was then charged trimethyl(trifluoromethyl)silane (77.3 g, 544 mmol) over the course of 1 hour at a rate which avoided temperature increase above 45° C. After an overnight stir without heating, the resultant mixture was transferred to a 2-neck round-bottom flask equipped with a magnetic stir bar and reflux condenser. The headspace of the flask was swept with a stream of N₂ to remove trimethylsilyl fluoride (TMS-F). The resultant mixture was used for subsequent synthetic transformations without purification.

Step 2a: Half of the mixture from Step 1 was transferred to a 250 mL round-bottom flask equipped with a magnetic stir bar and reflux condenser. With stirring, the reaction mixture was slowly heated to 60° C. followed by the dropwise addition of 2,2,2-trifluoroethyl nonafluorobutanesulfonate (49.7 g, 130 mmol). After an overnight stir at the same temperature, the resultant reaction mixture was diluted with water (150 mL) then transferred to a separatory funnel. Removal of the aqueous layer afforded a crude fluorochemical layer for which GC-FID analysis indicated approximately 99% conversion of the 2,2,2-trifluoroethyl nonafluorobutanesulfonate starting material.

Step 2b: Half of the mixture from Step 1 was transferred to a 250 mL round-bottom flask equipped with a magnetic stir bar and reflux condenser. With stirring, the reaction mixture was slowly heated to 60° C. followed by the dropwise addition of 2,2,2-trifluoroethyl trifluoromethanesulfonate (30.5 g, 130 mmol). After an overnight stir at the same temperature, the resultant reaction mixture was diluted with water (150 mL) then transferred to a separatory funnel. Removal of the aqueous layer afforded a crude fluorochemical layer for which GC-FID analysis indicated approximately 97% conversion of the 2,2,2-trifluoroethyl trifluoromethanesulfonate starting material.

The crude fluorochemical product mixtures from steps 2a and 2b were combined and purified via fractional distillation (90.9° C., 740 mm/Hg) to afford the desired 1,1,1,2,2,4,4,4-octafluoro-3-(2,2,2-trifluoroethoxy)-3-(trifluoromethyl)butane (58.4 g, 61% isolated yield) as a colorless liquid. The identity of the desired 1,1,1,2,2,4,4,4-octafluoro-3-(2,2,2-trifluoroethoxy)-3-(trifluoromethyl)butane was confirmed by GC-MS analysis.

### Example 16: 1,1,1,2,2,4,4,4-octafluoro-3-(2,2,3,3,3-pentafluoropropoxy)-3-(trifluoromethyl)butane

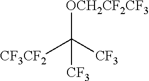

Step 1: To a 600 mL stainless steel reaction vessel equipped with an overhead stirrer were added tetraglyme (100 mL), potassium fluoride (19.2 g, 331 mmol), and 18-Crown-6 (8.0 g, 30.1 mmol). The reaction vessel was sealed and evacuated. To the vessel was then added 2,2,3,3,3-pentafluoropropanoyl fluoride (50.0 g, 301 mmol) in one portion. To the stirring mixture was then charged trimethyl(trifluoromethyl)silane (89.9 g, 632 mmol) over the course of 1 hour at a rate which avoided temperature increase above 45° C. After an overnight stir without heating, the resultant mixture was transferred to a 2-neck round-bottom flask equipped with a magnetic stir bar and reflux condenser. The headspace of the flask was swept with a stream of N₂ to remove trimethylsilyl fluoride (TMS-F). The resultant mixture was used for subsequent synthetic transformations without purification.

Step 2: One-third of the product mixture from Step 1 was transferred to a 3-neck round-bottom flask equipped with a magnetic stir bar, temperature probe, and reflux condenser. To the stirring, heated (45° C.) mixture was added 2,2,3,3,3-pentafluoropropyl trifluoromethanesulfonate (25 g, 89 mmol) dropwise over the course of 30 minutes. After a 2 day stir, the resultant mixture was diluted with water (100 mL) and then transferred to a separatory funnel. Removal of the aqueous layer afforded a crude fluorochemical layer for which GC-FID analysis indicated complete conversion of the 2,2,3,3,3-pentafluoropropyl trifluoromethanesulfonate starting material and 79% yield of the desired 1,1,1,2,2,4,4,4-octafluoro-3-(2,2,3,3,3-pentafluoropropoxy)-3-(trifluoromethyl)butane. Fractional distillation (109° C., 740 mm/Hg) afforded the desired 1,1,1,2,2,4,4,4-octafluoro-3-(2,2,3,3,3-pentafluoropropoxy)-3-(trifluoromethyl)butane (18.4 g, 50% isolated yield) The identity of the desired 1,1,1,2,2,4,4,4-octafluoro-3-(2,2,3,3,3-pentafluoropropoxy)-3-(trifluoromethyl)butane was confirmed by GC-MS analysis.

### Example 17: 1,1,1,2,2,4,4,4-octafluoro-3-(2,2,3,3,4,4,4-heptafluorobutoxy)-3-(trifluoromethyl)butane

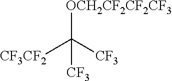

Step 1: To a 600 mL stainless steel reaction vessel equipped with an overhead stirrer were added tetraglyme (100 mL), potassium fluoride (16.1 g, 277 mmol), and 18-Crown-6 (10.5 g, 39.7 mmol). The reaction vessel was sealed and evacuated. To the vessel was then added 2,2,3,3,3-pentafluoropropanoyl fluoride (43.0 g, 259 mmol) in one portion. To the stirring mixture was then charged trimethyl(trifluoromethyl)silane (77.3 g, 544 mmol) over the course of 1 hour at a rate which avoided temperature increase above 45° C. After an overnight stir without heating, the resultant mixture was transferred to a 2-neck round-bottom flask equipped with a magnetic stir bar and reflux condenser. The headspace of the flask was swept with a stream of N₂ to remove trimethylsilyl fluoride (TMS-F). The resultant mixture was used for subsequent synthetic transformations without purification.

Step 2a: Half of the mixture from Step 1 was transferred to a 250 mL round-bottom flask equipped with a magnetic stir bar and reflux condenser. With stirring, the reaction mixture was slowly heated to 60° C. followed by the dropwise addition of 1H,1H-heptafluorobutyl nonafluorobutanesulfonate (62.7 g, 130 mmol). After an overnight stir at the same temperature, the reaction mixture was allowed to cool to room temperature and then diluted with water (150 mL). The mixture was transferred to a separatory funnel and the removal of the aqueous layer afforded a crude fluorochemical mixture for which GC-FID indicated approximately 73% yield of the desired 1,1,1,2,2,4,4,4-octafluoro-3-(2,2,3,3,4,4,4-heptafluorobutoxy)-3-(trifluoromethyl)butane. The crude fluorochemical mixture was purified via fractional distillation (134 C, 740 mm/Hg) afforded the desired 1,1,1,2,2,4,4,4-octafluoro-3-(2,2,3,3,4,4,4-heptafluorobutoxy)-3-(trifluoromethyl)butane (28.6 g, 47% isolated yield) as a colorless liquid. The identity of the desired 1,1,1,2,2,4,4,4-octafluoro-3-(2,2,3,3,4,4,4-heptafluorobutoxy)-3-(trifluoromethyl)butane was confirmed by GC-MS analysis.

### Example 18: 1,1,1,3,3,3-hexafluoro-2-(2,2,3,3,3-pentafluoropropoxy)-2-(trifluoromethyl)propane

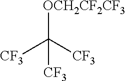

Step 1: To a 600 mL stainless steel reaction vessel equipped with an overhead stirrer were added N,N-dimethylformamide (105 mL) and potassium fluoride (20.1 g, 355 mmol). The reactor was then sealed and evacuated followed by the slow addition of 1,1,1,3,3,3-hexafluoropropan-2-one (50.1 g, 302 mmol). The reaction mixture was allowed to return back to 25° C. followed by the addition of trimethyl(trifluoromethyl)silane (47.2 g, 332 mmol) at a rate which avoided reaction mixture temperature increases above 30° C. After complete addition, the resultant mixture was allowed to stir overnight at 25° C. The mixture was then transferred to a 250 mL 3-neck round-bottom flask and slowly heated to 70° C. and the headspace of the flask was swept with a stream of N₂ to remove trimethylsilyl fluoride (TMS-F). The resultant mixture was used for subsequent synthetic transformations without purification.

Step 2: Half of the mixture from Step 1 was transferred to a 250 mL 3-neck round-bottom flask equipped with a magnetic stir bar, temperature probe, and reflux condenser. With stirring, the reaction mixture was slowly heated to 45° C. followed by the dropwise addition of 2,2,3,3,3-pentafluoropropyl 1,1,2,2,3,3,4,4,4-nonafluorobutane-1-sulfonate (65.1 g, 150.6 mmol). The resultant mixture was allowed to stir overnight at 60° C. The reaction mixture was allowed to cool to room temperature, diluted with water (150 mL), and then transferred to a separatory funnel. Removal of the aqueous layer afforded a crude fluorochemical mixture which was purified via fractional distillation (85° C., 740 mm/Hg) to afford the desired 1,1,1,3,3,3-hexafluoro-2-(2,2,3,3,3-pentafluoropropoxy)-2-(trifluoromethyl)propane (29.1 g, 53% isolated yield) as a colorless liquid. The identity of the desired 1,1,1,3,3,3-hexafluoro-2-(2,2,3,3,3-pentafluoropropoxy)-2-(trifluoromethyl)propane was confirmed by GC-MS analysis.

### Example 19: 1,1,1,2,2,3,3-heptafluoro-4-((1,1,1,3,3,3-hexafluoro-2-(trifluoromethyl)propan-2-yl)oxy)butane

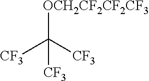

Step 1: To a 600 mL stainless steel reaction vessel equipped with an overhead stirrer were added N,N-dimethylformamide (105 mL) and potassium fluoride (20.1 g, 355 mmol). The reactor was then sealed and evacuated followed by the slow addition of 1,1,1,3,3,3-hexafluoropropan-2-one (50.1 g, 302 mmol). The reaction mixture was allowed to return back to 25° C. followed by the addition of trimethyl(trifluoromethyl)silane (47.2 g, 332 mmol) at a rate which avoided reaction mixture temperature increases above 30° C. After complete addition, the resultant mixture was allowed to stir overnight at 25° C. The mixture was then transferred to a 250 mL 3-neck round-bottom flask and slowly heated to 70° C. and the headspace of the flask was swept with a stream of N₂ to remove trimethylsilyl fluoride (TMS-F). The resultant mixture was used for subsequent synthetic transformations without purification.

Step 2: Half of the mixture from Step 1 was transferred to a 250 mL 3-neck round-bottom flask equipped with a magnetic stir bar, temperature probe, and reflux condenser. With stirring, the reaction mixture was slowly heated to 45° C. followed by the dropwise addition of 1H,1H-heptafluorobutyl nonafluorobutanesulfonate (72.5 g, 150 mmol). The resultant mixture was allowed to stir overnight at 60° C. The reaction mixture was allowed to cool to room temperature, diluted with water (150 mL), and then transferred to a separatory funnel. Removal of the aqueous layer afforded a crude fluorochemical mixture which was purified via fractional distillation (108° C., 740 mm/Hg) to afford the desired 1,1,1,2,2,3,3-heptafluoro-4-((1,1,1,3,3,3-hexafluoro-2-(trifluoromethyl)propan-2-yl)oxy)butane (30.2 g, 48% isolated yield) as a colorless liquid. The identity of the desired 1,1,1,2,2,3,3-heptafluoro-4-((1,1,1,3,3,3-hexafluoro-2-(trifluoromethyl)propan-2-yl)oxy)butane was confirmed by GC-MS analysis.

### Test Methods

Atmospheric lifetime: The atmospheric lifetime of hydrofluoroether Example 1 was determined from its rate of reaction with hydroxyl radicals. The pseudo-first order rate for the reaction of the gaseous 1,1,1,2,2,4,4,4-octafluoro-3-methoxy-3-(trifluoromethyl)butane with hydroxyl radical was measured in a series of experiments relative to reference compounds such as chloromethane and ethane. The measurements were performed in a 5.7 L, heated FTIR gas cell equipped with a polished semiconductor-grade quartz window. An Oriel Instruments UV Lamp, Model 66921 equipped with a 480 W mercury-xenon bulb was used to generate hydroxyl radicals by photolyzing ozone in the presence of water vapor. The concentrations of the hydrofluoroether and the reference compound were measured as a function of reaction time using an I-Series FTIR from Midac Corporation. The atmospheric lifetime was calculated from the reaction rates for the hydrofluoroether relative to the reference compounds and the reported lifetime of the reference compounds as shown below:

$${τ}_{x} = {τ}_{r} \cdot \frac{{k}_{r}}{{k}_{x}}$$

where τₓ is the atmospheric lifetime of hydrofluoroether, τᵣ is the atmospheric lifetime of the reference compound, and kₓ and kᵣ are the rate constants for the reaction of hydroxyl radical with the test compound and the reference compound, respectively.

Global Warming Potential (GWP): A measured IR cross-section was used to calculate the radiative forcing value for Example 1 (1,1,1,2,2,4,4,4-octafluoro-3-methoxy-3-(trifluoromethyl)butane) using the method of Pinnock et al. as described in J. Geophys. Res. 1995, 100, 23227-23238. The GWP (100-year iterative time horizon (ITH)) is calculated using the equation and methods described earlier in this specification, using the radiative forcing value and the experimentally determined atmospheric lifetime.

Specific heat capacity (Cₚ): Cₚ was measured using a TA Instruments Model Q2000 DSC (Differential Scanning Calorimeter) instrument. A sapphire standard was run before and after the samples and the average of the measured sapphire heat capacity vs. the theoretical heat capacity was used to correct the measured heat capacity of the sample.

Kinematic viscosity: Kinematic viscosity was measured using Schott-Ubbelohde Viscometers (glass capillary viscometers). The viscometers were timed using a viscometer timer available under the trade designation AVS-350 from SI Analytics, College Station, Tex., USA. The viscometer measurement stand and glass viscometer were immersed in a temperature-controlled liquid bath filled with NOVEC 7500 fluid available from 3M Company, Maplewood, Minn., USA. The temperature-controlled liquid bath (available from Lawler Manufacturing Corporation, Edison, N.J., USA), was fitted with a copper tubing coil for liquid nitrogen cooling with fine temperature control provided by the bath's electronic temperature control heater. The fluid was mechanically stirred to provide uniform 15 temperature in the bath. The bath controlled the temperature within +0.1° C., measured by a built-in RTD temperature sensor. The sample liquid was added to the viscometer between the two fill lines etched on the viscometer. The viscometer timer automatically pumped the sample fluid above the upper timing mark, then released the fluid and measured the efflux times between the upper and lower timing marks. The fluid meniscus was detected by optical sensors as it passed each timing mark. The sample was drawn up and measured repeatedly; results are provided as averages of multiple determinations. The glass viscometers were calibrated using certified kinematic viscosity standard fluids available from Cannon Instrument Company, State College, Pa., USA, to obtain a calibration constant (cSt/sec) for each viscometer. Kinematic viscosity in centiStokes (cSt) was calculated as the average efflux time (sec) times the viscometer calibration constant (cSt/sec).

Pour point: Pour point was determined visually and defined as the lowest temperature at which, after being tilted horizontally for 5 seconds, the sample was observed to flow. One to two milliliters of the sample were placed in a vial and cooled in a bath until it solidified. The sample was then allowed to warm slowly in the bath and observed every 3-5° C.

Largest Soluble Hydrocarbon (LSH): The LSH of each compound was determined by mixing the compound with hydrocarbons of varying molecular weight (CₙH₂ₙ₊₂, where n=9 to 14) in a hydrofluoroether:hydrocarbon ratio of about 1:1 to 1:2 by weight at room temperature (25° C.) and at 50° C. The LSH value is reported as the value of n in the formula CₙH₂ₙ₊₂ for the longest hydrocarbon which was compatible with the hydrofluoroether without exhibiting haze to the naked eye. A larger value of n is interpreted herein to indicate a greater ability of the hydrofluoroether to clean hydrocarbons.

Chemical Stability: Stability of compounds in the presence of the bases triethylamine (TEA), 1,4-diazabicyclo[2.2.2]octane (DABCO), and N,N,N,N-tetramethylethylene diamine (TMEDA) was tested as follows. To a 20 mL vial equipped with a magnetic stir bar were added the exemplary and comparative materials in the following starting amounts: Example 1-0.30 g (1.0 mmol), CE4-0.30 g (1.0 mmol) and CE5-0.30 g (1.0 mmol). FC-770 (0.40 g, 1.0 mmol) was added as an internal standard since it is known to be stable in the presence of the base materials tested herein. To this mixture was added one of: triethylamine (0.10 g, 1.0 mmol), DABCO (0.10 g, 0.89 mmol), or TMEDA (0.12 g, 1.0 mmol). The resultant mixture was stirred at 50° C. for 24 hours. GC-FID analysis was performed at specific time intervals to monitor the remaining hydrofluoroether and comparative materials present in the mixture.

Stability of compounds in the presence of N-methylpyrrolidinone (NMP) was tested as follows. To a 20 mL glass vial equipped with a magnetic stir bar was added 0.66 g of NMP (6.7 mmol) and one of the following exemplary or comparative materials in the following starting amounts: Example 1-2.0 g (6.7 mmol); CE4-2.0 g (6.7 mmol); CE5-2.0 g (6.7 mmol), or CE6-2.40 g (6.7 mmol). This mixture was then stirred at 50° C. for 144 h. To the resultant mixture was added H₂O (6.0 mL) and the aqueous layer was evaluated for fluoride ion content. The samples were analyzed using an Orion EA 940 meter with an Orion 9609BNWB Fluoride-ISE. Orion Ionplus Fluoride standards were used to calibrate the meter.

### Results

The atmospheric lifetime of Example 1 was determined from its rate of reaction with hydroxyl radicals as described above to yield a calculated atmospheric lifetime of 2.3 years. Using this value, the GWP (100-year iterative time horizon (ITH)) for Example 1 was found to be 170. This is much lower than the GWP of PFCs (including perfluorinated hydrocarbons, perfluorinated amines and perfluorinated ethers or polyethers) and lower than other closely related hydrofluoromethyl ethers.

Table 2 compares the specific heat capacity of Example 3 with commercially available heat transfer fluids. Given the similarity of the materials with respect to specific heat capacity, the compositions of the present disclosure could also serve as heat transfer fluids.

TABLE 2
Heat Capacity

| | Specific Heat |
|---|---|
| Material | Capacity (J/g · K) |
| Example 3 | 1.16 |
| CE1 | 1.05 |
| CE2 | 1.05 |
| CE3 | 1.10 |

The measured kinematic viscosity for Example 3 at various temperatures is shown in Table 3. A pour point of −62° C. was measured for Example 3. These results indicate that hydrofluoroethers of the present invention are suitable fluids for heat transfer and cleaning applications.

TABLE 3
Kinematic Viscosity for Example 3

| | Kinematic |
|---|---|
| Temp ° C. | Viscosity (cSt) |
| 0.0 | 0.97 |
| −10.0 | 1.65 |
| −20.0 | 2.15 |
| −30.0 | 2.88 |
| −40.0 | 5.19 |

Largest soluble hydrocarbon (LSH) values for Examples 1 and 2 at 25° C. and 50° C. are provided in Table 4. The results in Table 4 indicate that the hydrofluoroethers of the present invention are suitable fluids for cleaning applications.

TABLE 4
Largest Soluble Hydrocarbon

| | Largest Soluble Hydrocarbon | |
|---|---|---|
| | (LSH) | |
| Example | 25° C. | 50° C. |
| Ex. 1 | 9 | 11 |
| Ex. 2 | 11 | 13 |

Results of stability testing of Example 1, CE4, and CE5 in the presence of TEA, DABCO, and TMEDA are presented in Tables 5, 6, and 7, respectively. Tables 5-7 list the percentages (on a mole basis) remaining of each material after various exposure times, based on the initial amount. The low consumption of Example 1 in TEA, DABCO, and TMEDA indicates that the hydrofluoroethers of the present invention have exceptional stability in the presence of bases and are suitable fluids for cleaning applications.

TABLE 5
Stability with TEA

| | Mole % of Starting Material | | |
|---|---|---|---|
| Time (hr) | Example 1 | CE4 | CE5 |
| 0 | 100 | 100 | 100 |
| 16 | 100 | 100 | 100 |
| 24 | 100 | 96.7 | 86.7 |

TABLE 6
Stability with DABCO

| | Mole % of Starting Material | | |
|---|---|---|---|
| Time (hr) | Example 1 | CE4 | CE5 |
| 0 | 100 | 100 | 100 |
| 16 | 100 | 66.7 | 46.7 |
| 24 | 100 | 56.7 | 43.3 |

TABLE 7
Stability with TMEDA

| | Mole % of Starting Material | | |
|---|---|---|---|
| Time (hr) | Example 1 | CE4 | CE5 |
| 0 | 100 | 100 | 100 |
| 16 | 100 | 53.3 | 36.7 |
| 24 | 100 | 53.3 | 36.7 |

Results of stability testing of Example 1, CE4, CE5 and CE6 in the presence of NMP are summarized in Table 8. A low amount of fluoride is interpreted to mean that the test material is relatively stable. The results in Table 8 indicate that in comparison to CE4-CE6, the hydrofluoroethers of the present invention have exceptional stability in the presence of bases such as NMP and are suitable fluids for cleaning applications.

TABLE 8
Stability with NMP

| | Fluoride ion |
|---|---|
| Material | content (ppm) |
| Example 1 | 15.1 |
| CE4 | 388.8 |
| CE5 | 657.8 |
| CE6 | 49.3 |

Various modifications and alterations to this disclosure will become apparent to those skilled in the art without departing from the scope and spirit of this disclosure. It should be understood that this disclosure is not intended to be unduly limited by the illustrative embodiments and examples set forth herein and that such examples and embodiments are presented by way of example only with the scope of the disclosure intended to be limited only by the claims set forth herein as follows. All references cited in this disclosure are herein incorporated by reference in their entirety.

1. A compound having structural formula (II): 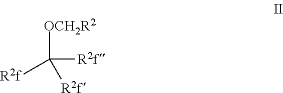
where R² is H, CH₃, CF₃, CH₂CF₂CF₂H, or CH₂CF₂CF₂CF₂CF₂H;
where R²f is a perfluoroalkyl group having 1-4 carbon atoms, optionally comprising either or both of a catenated nitrogen heteroatom and a catenated oxygen heteroatom;
where R²f′ is CF₃ or CF₂CF₃; and
where R²f″ is CF₃ or CF₂CF₃;
with the proviso that when R²f′ is CF₃, then R²f″ is CF₃, and when R² is H or CH₃, then R²f is not CF³.

2. The compound of claim 1, wherein R² is H or CH₃.

3. An apparatus for heat transfer comprising:
a device; and
a mechanism for transferring heat to or from the device, the mechanism comprising a working fluid that comprises a compound having structural formula (I): 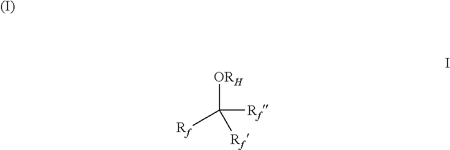
where R_{H} is CH₃, CH₂CH₃, or a partially fluorinated alkyl group having 1-5 carbon atoms;
where Rf is a perfluoroalkyl group having 1-9 carbon atoms, optionally comprising either or both of a catenated nitrogen heteroatom and a catenated oxygen heteroatom, and optionally comprising a 5- or 6-membered ring; and
where Rf′ and Rf″ are, independently, perfluoroalkyl groups having 1-2 carbon atoms.

4. The apparatus of claim 3, where R_{H} is CH₃ or CH₂CH₃.

5. The apparatus of claim 3, where R_{H} a partially fluorinated alkyl group having 1-2 carbon atoms.

6. The apparatus of claim 3, where Rf comprises either or both of a catenated nitrogen heteroatom and a catenated oxygen heteroatom.

7. The apparatus of claim 3, wherein the device is selected from a microprocessor, a semiconductor wafer used to manufacture a semiconductor device, a power control semiconductor, an electrochemical cell, a battery pack, an electrical distribution switch gear, a power transformer, a circuit board, a multi-chip module, a packaged or unpackaged semiconductor device, a fuel cell, and a laser.

8. The apparatus of claim 3, wherein the mechanism for transferring heat is a component in a system for maintaining a temperature or temperature range of the device.

9. A method of transferring heat comprising:
providing a device; and transferring heat to or from the device using a heat transfer fluid that comprises a compound having structural formula (I): 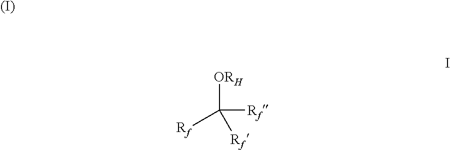
where R_{H} is CH₃, CH₂CH₃, or a partially fluorinated alkyl group having 1-5 carbon atoms;
where Rf is a perfluoroalkyl group having 1-9 carbon atoms, optionally comprising either or both of a catenated nitrogen heteroatom and a catenated oxygen heteroatom, and optionally comprising a 5- or 6-membered ring; and
where Rf′ and Rf″ are, independently, perfluoroalkyl groups having 1-2 carbon atoms.

10. A cleaning composition comprising:
a compound having structural formula (I): 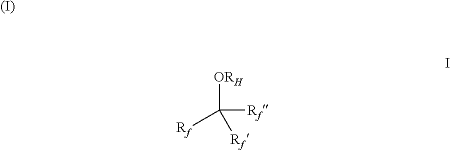
where R_{H} is CH₃, CH₂CH₃, or a partially fluorinated alkyl group having 1-5 carbon atoms;
where Rf is a perfluoroalkyl group having 1-9 carbon atoms, optionally comprising either or both of a catenated nitrogen heteroatom and a catenated oxygen heteroatom, and optionally comprising a 5- or 6-membered ring; and
where Rf′ and Rf″ are, independently, perfluoroalkyl groups having 1-2 carbon atoms; and
a co-solvent.

11. The cleaning composition of claim 10, wherein said compound having structural formula (I) is present in the composition in an amount of greater than 50 percent by weight, based on the total weights of the compound having structural formula (I) and the co-solvent.

12. The cleaning composition of claim 10, wherein said co-solvent comprises alcohols, ethers, alkanes, alkenes, haloalkenes, perfluorocarbons, perfluorinated tertiary amines, perfluoroethers, cycloalkanes, esters, ketones, oxiranes, aromatics, haloaromatics, siloxanes, hydrochlorocarbons, hydrochlorofluorocarbons, hydrofluorocarbons, hydrofluoroolefins, hydrochloroolefins, hydrochlorofluoroolefins, hydrofluoroethers, or mixtures thereof.

13. A cleaning composition comprising:
a compound having structural formula (I): 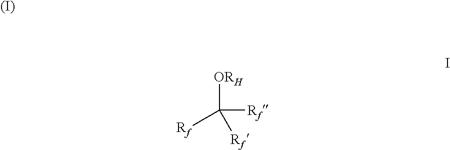
where R_{H} is CH₃, CH₂CH₃, or a partially fluorinated alkyl group having 1-5 carbon atoms;
where Rf is a perfluoroalkyl group having 1-9 carbon atoms, optionally comprising either or both of a catenated nitrogen heteroatom and a catenated oxygen heteroatom, and optionally comprising a 5- or 6-membered ring; and
where Rf′ and Rf″ are, independently, perfluoroalkyl groups having 1-2 carbon atoms; and
a surfactant.

14. The cleaning composition of claim 13, wherein the cleaning composition comprises from 0.1 to 5 percent by weight surfactant, based on the total weight of the composition.

15. The cleaning composition of claim 13, wherein the surfactant comprises a nonionic surfactant comprising an ethoxylated alcohol, an ethoxylated alkylphenol, an ethoxylated fatty acid, an alkylaryl sulfonate, a glycerolester, an ethoxylated fluoroalcohol, a fluorinated sulfonamide, or mixtures thereof.

16. A process for removing contaminants from a substrate, the process comprising the steps of:
contacting a contaminated substrate with the cleaning composition of claim 10.

17. An immersion cooling system comprising:
a housing having an interior space;
a heat-generating component disposed within the interior space; and
a working fluid liquid disposed within the interior space such that the heat-generating component is in contact with the working fluid liquid;
wherein the working fluid comprises a compound having structural formula (I): 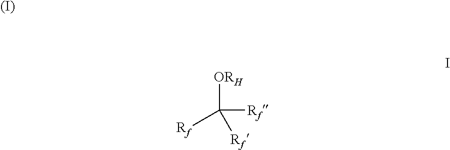
where R_{H} is CH₃, CH₂CH₃, or a partially fluorinated alkyl group having 1-5 carbon atoms;
where Rf is a perfluoroalkyl group having 1-9 carbon atoms, optionally comprising either or both of a catenated nitrogen heteroatom and a catenated oxygen heteroatom, and optionally comprising a 5- or 6-membered ring; and
where Rf′ and Rf″ are, independently, perfluoroalkyl groups having 1-2 carbon atoms.

18. The immersion cooling system of claim 17, wherein the compound is present in the working fluid at an amount of at least 25% by weight based on the total weight of the working fluid.

19. The immersion cooling system of claim 17, wherein the heat-generating component comprises an electronic device.

20. The immersion cooling system of claim 19, wherein the electronic device comprises a computer server.

21. A thermal management system for a lithium-ion battery pack comprising:
a lithium-ion battery pack; and
a working fluid in thermal communication with the lithium-ion battery pack;
wherein the working fluid comprises a compound having structural formula (I): 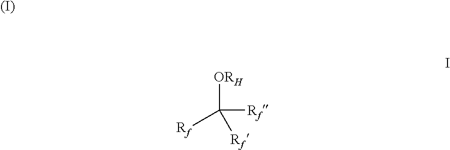
where R_{H} is CH₃, CH₂CH₃, or a partially fluorinated alkyl group having 1-5 carbon atoms;
where Rf is a perfluoroalkyl group having 1-9 carbon atoms, optionally comprising either or both of a catenated nitrogen heteroatom and a catenated oxygen heteroatom, and optionally comprising a 5- or 6-membered ring; and
where Rf′ and Rf″ are, independently, perfluoroalkyl groups having 1-2 carbon atoms.
